(19) United States
(12) Patent Application Publication
(10) Pub. No.: US 2011/0091536 A1
(43) Pub. Date: Apr. 21, 2011

(54) COMPOSITIONS COMPRISING EUPHORBIA PROSTRATA AND PROCESS OF PREPARATION THEREOF

(75) Inventors: Rajesh Jain, New Delhi (IN); Sukhjeet Singh, New Delhi (IN); Shilpa Bhasin, New Delhi (IN)

(73) Assignee: PANACEA BIOTEC LTD., New Delhi (IN)

(21) Appl. No.: 12/995,436

(22) PCT Filed: May 28, 2009
(86) PCT No.: PCT/IN2009/000308

§ 371 Date: Nov. 30, 2010

(30) Foreign Application Priority Data
May 30, 2008 (IN) 1312/DEL/2008

(51) Int. Cl. A61K 36/47; A61K 9/14; A61K 9/28; A61K 9/20; A61K 9/48; A61K 9/50; A61P 1/00; A61P 29/00

(52) U.S. Cl. 424/451; 424/725; 424/489; 424/474; 424/464; 424/490

(57) ABSTRACT

Oral pharmaceutical compositions comprising dry extract of Euphorbia prostrata from about 0.1% to about 99% by weight wherein the effective average particle size of the dry extract of Euphorbia prostrata is not more than about 500 microns with pharmaceutically acceptable excipient(s) and process(s) for the preparation of such compositions useful for the treatment of anorectal disease and colonic diseases such as hemorrhoids, fissures, cracks, fistulas, abscesses, inflammatory bowel disease and the like are provided. Further, the pharmaceutical compositions of the present invention provides for the administration of a therapeutically and/or prophylactically effective amount of the Euphorbia prostrata wherein the compositions possess properties to control inflammation, prevent capillary bleeding and fragility in mammalians, particularly human beings. Furthermore, the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s). Preferably the compositions of the present invention are in the form of tablets, minitablets, powders, capsules, pellets, granules, beads, compacts and the like. The present invention also provides and prophylactic and/or therapeutic methods of using such compositions for the treatment of anorectal diseases including hemorrhoids and colonic diseases.

### FIELD OF THE INVENTION

The present invention relates to oral pharmaceutical compositions comprising dry extract of Euphorbia prostrata from about 0.1% to about 99% by weight wherein the effective average particle size of the dry extract of Euphorbia prostrata is not more than about 500 microns with pharmaceutically acceptable excipient(s) and process(s) for the preparation of such compositions useful for the treatment of anorectal disease and colonic diseases such as hemorrhoids, fissures, cracks, fistulas, abscesses, inflammatory bowel disease and the like. Further, the pharmaceutical compositions of the present invention provides for the administration of a therapeutically and/or prophylactically effective amount of the Euphorbia prostrata wherein the compositions possess properties to control inflammation, prevent capillary bleeding and fragility in mammalians, particularly human beings. Furthermore, the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s). Preferably the compositions of the present invention are in the form of tablets, minitablets, powders, capsules, pellets, granules, beads, compacts and the like. The present invention also provides and prophylactic and/or therapeutic methods of using such compositions for the treatment of anorectal diseases including hemorrhoids and colonic diseases.

### BACKGROUND OF THE INVENTION

Among the various anorectal and colonic diseases, hemorrhoids occupy a prominent position and have been the subject of numerous clinical studies. Hemorrhoidal disease is characterized by bleeding, without any pain. Fresh blood spots occur immediately, on defecation. However, pain occurs when the hemorrhoids are secondarily infected, or complicated by thrombosis and anal fissures. Hemorrhoids can be caused by a variety of factors including hormones, genes, inflammation, infection, constipation, exercise, vascular stasis, diet, strain, physical stance in defecation, loss of connective tissue elasticity with age etc. The symptoms most widely recognized are bleeding, pain and prolapse (Hyams and Philpot, 1970; Smith, 1987). These may be accompanied by thrombosis, pruritis, edema etc. Hemorrhoids can be treated through reduction of inflammation and pain, hemostasis, wound healing and protection of vascular walls. Thus, an effective treatment of acute hemorrhoidal attacks should not only provide relief as early as 2-3 days after initiation of the treatment, but also reduce the recurrence of such attacks.

There exist several procedures for the treatment of hemorrhoids. PCT publication WO8803398 discloses surgical dressings for such treatment. Patents have been granted in respect of surgical devices such as European patent no. 0095142, U.S. Pat. No. 4,621,635 has been granted for the use of lasers in the treatment of hemorrhoids. The techniques of cryopharmacotherapy and electrochemical techniques for treatment of hemorrhoids have also been patented vide European patent no. 0091405 and European patent no. 0116688, respectively. However, the biggest drawbacks of the above are the involvement of medical experts beyond mere prescription of medicines and probable hospitalization. Also, some of them are physically and/or psychologically unpleasant in application for treating such diseases.

Several patents (U.S. Pat. Nos. 4,160,148, 4,508,728, 4,797,392, 4,518,583 and 5,234,914) have been granted in respect of compositions containing certain wound healing agents to provide symptomatic relief, by promoting tissue repair, reducing inflammation and encouraging wound healing. Some of them like U.S. Pat. Nos. 4,518,583 and 5,234,914 contain antimicrobial agents. These compositions, however, only relieve symptoms associated with inflammation, like heat, itching, redness, pain and swelling. A number of compositions for the treatment of anorectal diseases (including hemorrhoids) are based on the anesthetic and vasoconstrictive properties of the constituents, but these provide only temporary symptomatic relief.

Patents in the USA (U.S. Pat. Nos. 4,613,498; 4,626,433; 5,166,132; 5,219,880; 5,234,914 and 4,797,392) and Europe (European patents nos. 0225832 and 0513442) have been granted in respect of compositions with varying constituents, for topical application in the form of suitable and acceptable pharmaceutical carriers, such as salts, ointments, etc., with organic, inorganic or biological active agents. However, these compositions provide only temporary relief and are limited to local application and cannot be used for systemic use or oral administration. A topical treatment for hemorrhoidal pain and for spasms of the sphincters and muscles located in the GI tract is disclosed in a granted patent (U.S. Pat. No. 5,595,753), which includes amino acid L-arginine in a pharmaceutically acceptable carrier. Another U.S. Pat. No. 5,591,436 has been granted for a composition for dietary supplement for the treatment of hemorrhoids. The composition comprises 60% to 95% Indian Barberry by weight; 4.8% to 38% Nagkesar by weight; and 0.2% to 2% Margosa tree leaves by weight. Another U.S. Pat. No. 5,562,906 discloses the use of bark or berries of the species Xanthoxylum clava herculis L and Xanthoxylum americanum Hill, both of the yellow wood tree family, are employed for the treatment of hemorrhoids and other membrane and capillary disorders of the veins and arteries. Improved strength and flexibility of the veins, arteries and their constituent structures is obtained.

An Indian patent no. 186803 and several other patents (Australia, No. 698407; China, No. CN 1102387C; Europe, No. 868914; Russia, No. 2174396; South Africa, No. 97/2900; South Korea, No. 281679 and U.S., U.S. Pat. No. 5,858,371) have been granted to the present applicant for a composition comprising a flavonoid containing extract of Euphorbia prostrata for treatment of anorectal and colonic diseases. Recently a U.S. Pat. No. 7,371,412 has been granted to the applicants of the present invention for a composition comprising of an extract of the plant Euphorbia prostrata, particularly with pharmaceutically acceptable carrier(s)/base(s), optionally with additional therapeutic agent(s) useful for the treatment of anorectal disease and colonic diseases such as hemorrhoids, fissures, cracks, fistulas, abscesses, inflammatory bowel disease, and the like.

Nowhere in the prior art is disclosed oral pharmaceutical compositions comprising dry extract of Euphorbia prostrata from about 0.1% to about 99% by weight wherein the effective average particle size of the dry extract of Euphorbia prostrata or drug or active agent is not more than about 500 microns with pharmaceutically acceptable excipient(s) wherein the compositions preferably provides a release of not less than about 50% of the dry extract of Euphorbia prostrata or drug or active agent in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s). The inventors with expenditure of intellectual effort and careful experimentation have prepared oral pharmaceutical compositions for the long-term management of anorectal diseases including hemorrhoids, and colonic diseases that are safe and painless to administer and have long-term effectiveness. The compositions of the present invention have improved efficacy and safety and are economical to manufacture.

### SUMMARY OF THE INVENTION

It is an objective of the present invention to provide oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 500 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

The phrase ‘dry extract of Euphorbia prostrata’ as herein described is prepared by drying the plant Euphorbia prostrata under controlled conditions of temperature and humidity, making a powder from the dried plant, extracting the dry coarse powder with a polar solvent repetitively to form an extract, distilling the extract, optionally washing the concentrated extract with a non-polar organic solvent, optionally re-extracting the concentrated extract with a medium polarity organic solvent and drying the washed extract to produce the desired pharmaceutically acceptable extract.

It is an objective of the present invention to provide oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 350 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

It is an objective of the present invention to provide oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 250 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

It is an objective of the present invention to provide to provide process for the preparation of oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 500 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s) as herein described, comprising of the following steps:
i) drying the extract to produce the desired pharmaceutically acceptable extract,
ii) mixing the dried extract obtained from step (i) with pharmaceutically acceptable excipient(s),
iii) formulating the mixture obtained in step (ii) into a suitable dosage form.

Yet another objective of the present invention is to provide method of using such pharmaceutical compositions for the treatment of anorectal or colonic disease such as hemorrhoids, fissures, cracks, fistulas, abscesses, and inflammatory bowel disease.

The compositions of the present invention and method for treating anorectal diseases including hemorrhoids and colonic diseases using dry extract of Euphorbia prostrata provides long-term effectiveness and low prolapse rates.

### DETAILED DESCRIPTION OF THE INVENTION

The present invention provides compositions that can be orally administered. The invention provides relief from pain associated with hemorrhoids. The invention also significantly reduces bleeding and accelerates tissue re-growth in the affected hemorrhoidal tissue. The invention is useful in the treatment of lesions, other than hemorrhoids in the anorectal area and can be formulated in several types of dosage forms. There are no side effects from the use of the composition in human beings. The plant Euphorbia prostrata (Family: Euphorbiaceae) was identified as being relevant in the study of anorectal and colonic diseases, including hemorrhoids. Euphorbia prostrata is well known to the Indian traditional medicine in the use of treatment for asthma, bloody dysentery, and sores.

In an embodiment of the present invention is provided an oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 500 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

The phrase ‘dry extract of Euphorbia prostrata’ as herein described is prepared by drying the plant Euphorbia prostrata under controlled conditions of temperature and humidity, making a powder from the dried plant, extracting the dry coarse powder with a polar solvent repetitively to form an extract, distilling the extract, optionally washing the concentrated extract with a non-polar organic solvent, optionally re-extracting the concentrated extract with a medium polarity organic solvent and drying the washed extract to produce the desired pharmaceutically acceptable extract.

In another embodiment of the present invention is provided an oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 350 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

In an embodiment of the present invention is provided an oral pharmaceutical composition for the treatment of anorectal or colonic disease such as hemorrhoids, fissures, cracks, fistulas, abscesses, inflammatory bowel disease, and the like comprising dry extract of Euphorbia prostrata from about 0.1% to about 99% by weight wherein the effective average particle size of the dry extract of Euphorbia prostrata is not more than about 250 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

The pharmaceutical composition into different dosage forms can be formulated using pharmaceutically acceptable excipient(s) and techniques known to art. Pharmaceutical dosage forms of the present invention can be in the form selected from but not limited to tablets (coated or uncoated), chewable tablets, mini-tablets, troches and lozenges, sachet, capsules (hard or soft), mini-tablets/tablets filled in capsule, granules, solutions, suspensions, powder, sublingual dosage forms, wafers, caplets, and other dosage forms suitable for oral administration. In an embodiment, the compositions can be prepared by dissolving or dispersing dry extract of Euphorbia prostrata in a solvent or mixture of solvents with or without pharmaceutically acceptable excipients and depositing or layering or spraying the solution or dispersion onto inert beads, spheres, cores, seeds, particles or nuclei. Useful inert beads, spheres, cores, seeds, particles or nuclei can comprise of but not limited to water-soluble materials such as sugar spheres, lactose and the like, and mixtures thereof without limitation thereto. The inert beads, spheres, cores, seeds, particles, or nuclei can also comprise of but not limited to water-insoluble materials such as celluloses such as microcrystalline cellulose spheres, glass beads; plastic particles; water-insoluble or partially soluble inorganic materials such as calcium carbonate, dicalcium phosphate anhydrous, dicalcium phosphate monohydrate, tribasic calcium phosphate, magnesium carbonate, and magnesium oxide; and the like and mixtures thereof without limitation thereto. In a preferred embodiment of the present invention, the composition is formulated in the form of tablets. The tablets can be prepared by either direct compression, dry compression (slugging) or by wet granulation. In a preferred embodiment of the present invention, the oral composition is prepared by direct compression/compaction, slugging, extrusion, molding, or the like using excipients known to the person skilled in the art. The composition may also be prepared by wet granulation technique, which may use either aqueous/polar solvent(s) or non-aqueous/organic solvent(s) or mixtures thereof. The non-aqueous solvent used is selected from but not limited to a group comprising ethanol, isopropyl alcohol, methylene chloride or acetone. In an embodiment, the compositions of the present invention may be in the form of compacted tablets/mini-tablets, compressed tablets/mini-tablets, or moulded tablets/mini-tablets, coated tablets/mini-tablets and the like. In another embodiment, the coated tablets/mini-tablets may optionally comprise a part or whole of the active agent in the coating composition. In an embodiment of the present invention, a suitable organic solvent system is selected from but not limited to, methanol, ethanol, 1-butanol, 2-butanol, 3-methyl-1-butanol, 1-propanol, 2-propanol, isopropanol, 1-pentanol, acetone, methyl acetate, ethyl acetate, butyl acetate, propyl acetate, isopropyl acetate, isobutyl acetate, ethyl ether, tert-butylmethyl ether, ethyl formate, chloroform, dichloromethane, and the like or mixtures thereof.

In an embodiment, the polar solvent(s) used in the present invention for the process of preparation of dry extract of Euphorbia prostrata is selected from but not limited to a group comprising acetone, methanol, ethanol, isopropanol, water, and the like used either alone or in combination thereof. In a further embodiment, the non-polar organic solvent(s) used in the present invention for the process of preparation of dry extract of Euphorbia prostrata is selected from but not limited to a group comprising pentane, hexane, heptane, petroleum ether, chloroform, dichloromethane, dichloroethane, and the like used either alone or in combination thereof. In an embodiment the medium polarity organic solvent(s) is selected from but not limited to ethyl acetate, ethyl methyl ketone, butanol, and the like used either alone or in combination thereof.

The pharmaceutically acceptable excipient(s) used in the composition of the present invention are selected from but not limited to a group of excipients generally known to persons skilled in the art e.g. diluents such as lactose, mannitol, sorbitol, starch, microcrystalline cellulose, xylitol, fructose, sucrose, dextrose, dicalcium phosphate, calcium sulphate; disintegrants; binders; fillers; bulking agent such as ispaghula husk; organic acids; colorants; stabilizers; preservatives; lubricants; glidants; chelating agents; vehicles; bulking agents; stabilizers; preservatives; solubility enhancing agents such as glycerin, various grades of polyethylene oxides, transcutol and glycofurol; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof. The disintegrants used in the present invention include but not limited to starch or its derivatives, fully pregelatinized maize starch (Starch 1500®), croscarmellose sodium, sodium starch glycollate, and the like used either alone or in combination thereof. The binders used in the present in invention is selected from but not limited to a group comprising polyvinyl pyrrolidone, starch or its derivatives, fully pregelatinized maize starch, hydroxypropyl methyl cellulose, hydroxypropyl cellulose, gum arabic powder, gelatin and the like used alone or in combination thereof. The lubricants used in the present invention include but not limited to talc, magnesium stearate, calcium stearate, stearic acid, hydrogenated vegetable oil, sodium starch fumarate and the like used either alone or in combination thereof.

The capsules comprise from about 25 to about 300 mg of the dry extract of Euphorbia prostrata, preferably from about 50 to about 100 mg along with pharmaceutical excipients. Similarly, tablets may be prepared by dispersing from about 25 to about 300 mg of dry extract of Euphorbia prostrata, preferably from about 50 to about 100 mg along with other pharmaceutical excipients. In an embodiment, the tablets may be coated or uncoated.

In an embodiment of the present invention, the capsule may be taken, subject to a maximum of about 300 mg of dry extract or drug or active agent per day. In another embodiment, the granules in ready dispersible and effervescent form are prepared by using excipients such as sucrose, mannitol, sodium bicarbonate, citric acid, and the like.

In another embodiment, the composition is formulated as a film coated dosage form. The coating material used for film coating is selected from but not limited to the conventionally used materials such as cellulosic polymers, or the like or mixtures thereof. Other conventionally used excipients for the coating includes but not limited to plasticizers, solvents, lubricants, surfactants, and the like or mixtures thereof.

In an embodiment of the present invention, is provided a process for the preparation of an oral pharmaceutical composition for the treatment of anorectal or colonic disease such as hemorrhoids, fissures, cracks, fistulas, abscesses and inflammatory bowel disease comprising dry extract of Euphorbia prostrata with pharmaceutically acceptable excipient(s) as herein described, comprising of the following steps:
i) drying the extract to produce the desired pharmaceutically acceptable extract,
ii) mixing the dried extract obtained from step (i) with pharmaceutically acceptable excipient(s),
iii) formulating the mixture obtained in step (ii) into a suitable dosage form.

### Dissolution Study Method

The dissolution study method (I) in accordance with the present invention has the following parameters:
Dissolution medium: 0.5% w/v sodium lauryl sulphate in distilled water
Dissolution medium volume: 1000 ml
Apparatus: Paddle (USP Type II)
Paddle Speed: 50 rpm
Dissolution time: 15, 60 minutes

In an embodiment, the dissolution profile of the composition as described in Example-1 hereinafter comprising dry extract of Euphorbia prostrata is as follows:

| | | Dissolution profile |
|---|---|---|
| S. No. | Time (minutes) | (% drug release) |
| 1 | 10 | 75.5 |
| 2 | 15 | 80.5 |
| 3 | 30 | 82.3 |
| 4 | 45 | 83.7 |
| 5 | 60 | 85.5 |

In an embodiment of the present invention, a method of treatment of anorectal or colonic disease such as hemorrhoids, fissures, cracks, fistulas, abscesses, inflammatory bowel disease, and the like, comprising a dry extract of Euphorbia prostrata with pharmaceutically acceptable excipient(s) is provided.

In a further embodiment, use of a dry extract of the plant Euphorbia prostrata for the preparation of a pharmaceutical composition for the treatment of anorectal or colonic disease such as hemorrhoids, fissures, cracks, fistulas, abscesses, inflammatory bowel disease, and the like are provided.

The compositions of the present invention and method for treating anorectal diseases including hemorrhoids, and colonic diseases using a dry extract of Euphorbia prostrata provides long-term effectiveness and low prolapse rates. The treatment includes administration by oral route an effective amount of composition comprising of a pharmaceutically acceptable carrier and mixture of flavonoids and phenolic compounds extracted from Euphorbia prostrata.

The examples given below serve to illustrate embodiments of the present invention. However they do not intend to limit the scope of present invention in any manner whatsoever.

### EXAMPLE 1

| S. No. | Ingredient | mg/tablet |
|---|---|---|
| 1. | Dry extract of Euphorbia prostrata | 100 |
| | (effective average particle size is | |
| | not more than 500 microns) | |
| 2. | Mannitol | 348.85 |
| 3. | Microcrystalline cellulose | 41 |
| 4. | Pregelatinized starch | 5.5 |
| 5. | Purified water | (q.s.) Lost in processing |
| 6. | Croscarmellose sodium | 10 |
| 7. | Glycerol dibehenate | 3 |
| 8. | Colloidal silica, anhydrous | 3 |
| 9. | Magnesium stearate | 1.65 |
| 10. | Crospovidone | 37 |

Base coating

| | | |
|---|---|---|
| 11. | Opadry II | 13.75 |
| 12. | Purified water | (q.s.) Lost in processing |

### Procedure:

i) Dry extract of Euphorbia prostrata, microcrystalline cellulose and mannitol were sifted from sieve of mesh size #30 and mixed together.
ii) Pregelatinized starch was dispersed in purified water with continuous stirring to prepare the binder solution.
iii) The binder solution of step (ii) was mixed with the material of step (i) to obtain the granules followed by drying of the granules.
iv) Croscarmellose sodium, glycerol dibehenate, colloidal silicon anhydrous, magnesium stearate, crospovidone were sifted together through a sieve of mesh size 30.
v) The dried granules of step (iii) and the material of step (iv) was blended together for 10-12 minutes.
vi) The blended granules of step (v) were compressed into tablets.
vii) Opadry II was mixed in purified water for about 45 minutes to prepare the base coating solution.
viii) The core tablets of step (vi) were coated with the base coating solution obtained from step (vii).

### EXAMPLE 2

| S. No. | Ingredient | mg/tablet |
|---|---|---|
| 1. | Dry extract of Euphorbia prostrata | 100 |
| | (effective average particle size is | |
| | not more than 500 microns) | |
| 2. | Lactose | 340 |
| 3. | Microcrystalline cellulose | 45 |
| 4. | Polyvinyl pyrrolidone | 5 |
| 5. | Purified water | (q.s.) Lost in processing |
| 6. | Sodium starch glycollate | 10 |
| 7. | Magnesium stearate | 3.5 |
| 8. | Colloidal silica, anhydrous | 3 |
| 9. | Stearic acid | 1.5 |
| 10. | Crospovidone | 35 |

Base coating

| | | |
|---|---|---|
| 11. | Opadry II | 13.75 |
| 12. | Purified water | (q.s.) Lost in processing |

Film coating

| | | |
|---|---|---|
| 13. | Opadry AMB | 19.73 |
| 14. | Purified water | (q.s.) Lost in processing |

### Procedure:

i) Dry extract of Euphorbia prostrata, lactose and microcrystalline cellulose were sifted from sieve of mesh size #30 and mixed together.
ii) Polyvinyl pyrrolidone was dispersed in purified water with continuous stifling to prepare the binder solution.
iii) The binder solution of step (ii) was mixed with the material of step (i) to obtain the granules followed by drying of the granules.
iv) Sodium starch glycollate, magnesium stearate, colloidal silicon anhydrous, stearic acid, crospovidone were sifted together through a sieve of mesh size 30.
v) The dried granules of step (iii) and the material of step (iv) was blended together for 10-12 minutes.
vi) The blended granules of step (v) were compressed into tablets.
vii) Opadry II was mixed in purified water for about 45 minutes to prepare the base coating solution.
viii) The core tablets of step (vi) were coated with the base coating solution obtained from step (vii).
ix) Opadry AMB was mixed in purified water for about 45 minutes to prepare the final coating solution.
x) The base coated tablets of step (viii) were coated with the film coating solution obtained from step (ix).

### EXAMPLE 3

| S. No. | Ingredient | mg/capsule |
|---|---|---|
| 1. | Dry extract of Euphorbia prostrata | 100 |
| | (effective average particle size is | |
| | not more than 350 microns) | |
| 2. | Microcrystalline cellulose | 200.8 |
| 3. | Mannitol | 72 |
| 4. | Talc | 3.2 |
| 5. | Sodium starch glycollate | 12 |
| 6. | Colloidal silicon dioxide | 12 |

### Procedure:

i) Dry extract of Euphorbia prostrata, microcrystalline cellulose and mannitol were sifted from sieve of mesh size #30 and mixed together.
ii) Talc, sodium starch glycollate and colloidal silicon dioxide were passed through fine sieves individually and then mixed together.
iii) The materials of step (i) and (ii) were mixed.
iv) The material of step (iii) was filled into empty hard gelatin capsules at an average fill weight of 400 mg±2%.
v) The filled capsules were packed in air-tight packages.

1. An oral pharmaceutical composition, comprising dry extract of Euphorbia prostrata from about 0.1% to about 99% by weight wherein the effective average particle size of the dry extract of Euphorbia prostrata is not more than about 500 microns comprising flavonoids from about 3% to about 9% by weight, phenolic compounds from about 10% to about 50% by weight and other compounds from about 41% to about 87% by weight, wherein the flavonoids comprise apigenin-7-glycoside, 1-5% by weight; luteolin-7-glycoside, 0.3-4% by weight; and apigenin, luteolin and quercetin, 0.01-1% by weight and the like; and wherein the phenolic compounds comprise ellagic acid, 1-15% by weight; gallic acid, 1-12% by weight and tannins, 1-10% by weight and the like with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

2. The composition according to claim 1, wherein the effective average particle size of the dry extract of Euphorbia prostrata is not more than about 350 microns with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

3. The composition according to claim 1, wherein the effective average particle size of the dry extract of Euphorbia prostrata is not more than about 250 microns with pharmaceutically acceptable excipient(s); and wherein the compositions preferably provides a release of not less than about 50% of the dry extract of Euphorbia prostrata in first 15 minutes and not less than about 80% of dry extract of Euphorbia prostrata after total dissolution study of about 60 minutes when tested in accordance with the dissolution method described herein employing physiological pH range of about 2 to about 7.5 optionally with surfactant(s).

4. The pharmaceutical composition according to claim 1, which can be formulated into different dosage forms using pharmaceutically acceptable excipient(s) selected from tablets (coated or uncoated), chewable tablets, mini-tablets, troches and lozenges, sachet, capsules (hard or soft), mini-tablets/tablets filled in capsule, granules, solutions, suspensions, powder, sublingual dosage forms, wafers, caplets, and other dosage forms suitable for oral administration.

5. The composition according to claim 1, wherein the compositions can be prepared by dissolving or dispersing dry extract of Euphorbia prostrata in a solvent or mixture of solvents with or without pharmaceutically acceptable excipients and depositing or layering or spraying the solution or dispersion onto inert beads, spheres, cores, seeds, particles or nuclei.

6. The composition according to claim 5, wherein the inert beads, spheres, cores, seeds, particles or nuclei can comprise of water-soluble materials such as sugar spheres, lactose and the like, and mixtures thereof and water-insoluble materials such as celluloses such as microcrystalline cellulose spheres, glass beads; plastic particles; water-insoluble or partially soluble inorganic materials such as calcium carbonate, dicalcium phosphate anhydrous, dicalcium phosphate monohydrate, tribasic calcium phosphate, magnesium carbonate, and magnesium oxide; and the like and mixtures thereof.

7. The composition according to claim 4, wherein the composition is formulated in the form of compacted tablets/mini-tablets, compressed tablets/mini-tablets, or moulded tablets/mini-tablets, coated tablets/mini-tablets and the like.

8. The composition according to claim 7, wherein the coated tablets/mini-tablets may optionally comprise a part or whole of the active agent in the coating composition.

9. The compositions according to claim 1, wherein the compositions of the present invention comprises one or more pharmaceutically acceptable excipient(s) selected from a group comprising diluents; disintegrants; binders; fillers; bulking agent; organic acids; colorants; stabilizers; preservatives; lubricants; glidants; chelating agents; vehicles; bulking agents; stabilizers; preservatives; solubility enhancing agents; pH adjusting agents; antioxidants; osmotic agents; chelating agents; viscosifying agents; wetting agents; emulsifying agents; acids; sugar alcohol; reducing sugars; non-reducing sugars and the like used either alone or in combination thereof.

10. A process for the preparation of an oral pharmaceutical composition according to claim 1, for the treatment of anorectal or colonic disease such as hemorrhoids, fissures, cracks, fistulas, abscesses and inflammatory bowel disease comprising dry extract of Euphorbia prostrata with pharmaceutically acceptable excipient(s) as herein described, comprising of the following steps:
i). drying the extract to produce the desired pharmaceutically acceptable extract,
ii). mixing the dried extract obtained from step (i) with pharmaceutically acceptable excipient(s),
iii). formulating the mixture obtained in step (ii) into a suitable dosage form.

11. A method of using the pharmaceutical composition according to claim 1, which comprises administering to a patient in need thereof an effective amount of the composition.

12. A method of using the pharmaceutical composition according to claim 11, for the treatment of anorectal or colonic disease such as hemorrhoids, fissures, cracks, fistulas, abscesses, inflammatory bowel disease, and the like, comprising a dry extract of Euphorbia prostrata or drug or active agent with pharmaceutically acceptable excipient(s).

13. Use of a composition according to claim 1, for the preparation of medicament for the management of anorectal or colonic disease such as hemorrhoids, fissures, cracks, fistulas, abscesses, inflammatory bowel disease.

14. The pharmaceutical compositions and the processes for the preparation of pharmaceutical compositions substantially as herein described and illustrated by the examples.
